(19) United States
(12) Patent Application Publication
(10) Pub. No.: US 2025/0241669 A1
(43) Pub. Date: Jul. 31, 2025

(54) Stone Removal Device

(71) Applicant: SILVER BELL ENTERPRISE INC., Taipei City (TW)

(72) Inventor: Hsein-Jar Chiang, Zhubei City (TW)

(21) Appl. No.: 19/038,123

(22) Filed: Jan. 27, 2025

(30) Foreign Application Priority Data
Jan. 31, 2024 (TW) 113201189

(51) Int. Cl. A61B 17/221; A61B 17/00; A61B 17/22

(52) U.S. Cl. CPC A61B 17/221; A61B 17/00234; A61B 2017/00238; A61B 2017/00477; A61B 2017/00836; A61B 2017/00929; A61B 2017/00942; A61B 2017/00946; A61B 2017/22079; A61B 2017/2212; A61B 2217/005

(57) ABSTRACT

A stone removal device, comprising an outer tube, an inner tube configured to be sleeved within the outer tube, and a stone removal basket or a stone-crushing rod configured to be sleeved within the inner tube. The stone removal device is inserted into a bile duct or gallbladder of a patient via an expanded drainage channel. The stone removal basket is placed at the location of stones through the inner tube and the outer tube, and the stones are captured within the stone removal basket and the stone removal basket containing the stone is pulled outward to crush the stone while the outer tube and the inner tube are fixed. If the stone cannot be crushed by the stone removal basket, the stone-crushing rod can be used to break the stone into fragments. The stone fragments can then be removed from the body using the stone removal basket.

### FIELD OF INVENTION

The present invention is related to a stone removal device, particularly to a stone removal device for retrieving and removing calculus of bile duct from patient body in medical use.

### BACKGROUND OF THE INVENTION

In the current medical practice, removing calculus of intrahepatic duct include mainly three methods as followings.

1. Surgeon firstly incises a bile duct, inserts a choledochoscope into an intrahepatic duct, and then removes the stones. Due to excessive curvature or narrowing of the intrahepatic duct, this first approach is often impossible to remove the stones in most of the time and the patient suffers from the open wound surgery.

2. Second, radiologist punctures the intrahepatic duct and expand the channel. After waiting for six weeks until the punctured channel wall completes fibrosis, surgeon then inserts a choledochoscope into the intrahepatic duct to remove the stones. However, the same condition of the previous approach that due to excessive curvature of the intrahepatic duct or the even the size of the stones are too big, the stones often cannot be removed easily.

3. The third approach is for stones that occurring in a left intrahepatic duct. Surgeon normally will choose to remove the left lobe of the liver.

On the other hand, for removing common bile duct stones, there are two methods for current practice as following:

1. The first approach is that surgeon firstly incises the common bile duct, inserts a choledochoscope into the bile duct, and removes the stones directly in most of the condition.

2. Gastroenterologist uses a duodenoscope and an electrosurgical knife to incise the opening of the common bile duct, then insert a stone removal basket into the common bile duct to remove the stones. However, the stones are difficult to be removed if the stones are too large.

For gallbladder stones, the main treatment is laparoscopic cholecystectomy which is removing the gallbladder completely. However, for younger patients, removing the gallbladder at a young age can have long-term health implications over the course of their lives.

The above-mentioned stone removal methods require patients to undergo general anesthesia. Patients with cardiopulmonary diseases who cannot tolerate general anesthesia are unable to undergo such stone removal surgeries.

Hence, it is eager to have a solution that will overcome or substantially ameliorate at least one or more of the deficiencies of a prior art, or to at least provide an alternative solution to the problems. It is to be understood that, if any prior art information is referred to herein, such reference does not constitute an admission that the information forms part of the common general knowledge in the art.

### SUMMARY OF THE INVENTION

In order to solve shortcomings of the current treatment for gallbladder stones, which involves the complete removal of the gallbladder and may have long-term health implications for younger patients over the course of their lives, as well as the issue where patients with cardiopulmonary diseases who cannot undergo general anesthesia are unable to receive stone removal surgery, the present invention provides a stone removal device. The said device has an outer tube, an inner tube configured to be sleeved within the outer tube, and a stone removal basket and/or a stone-crushing rod configured to be sleeved within the inner tube; wherein the outer tube comprises an outer tube connector and an outer tube body detachably fixed to the outer tube connector.

The outer tube connector includes a first outer tube opening at its left end and a second outer tube opening at its right end, the first outer tube opening and the second outer tube opening are connected by an outer tube column, and an outer tube thread is provided on the outer wall of the first outer tube opening.

The outer tube body is a long slender tube with an outer tube connector engagement end at its left end, extending longitudinally to the outer tube body outlet at its right end, the outer diameter of the outer tube connector engagement end is slightly smaller than the inner diameter of the outer tube inner passage within the second outer tube opening of the outer tube column, allowing it to be sleeved and secured therein.

The inner tube comprises an inner tube connector and an inner tube body detachably fixed to the inner tube connector.

The inner tube connector includes a first inner tube opening at its left end and a second inner tube opening at its right end, the first inner tube opening extends towards the second inner tube opening, forming an inner tube column, an inner tube thread is provided on the outer wall of the first inner tube opening, the inner tube column of the second inner tube opening is configured to be sleeved within the first outer tube opening of the outer tube connector.

The inner tube body is a long slender tube with an inner tube connector engagement end at its left end, extending longitudinally to the inner tube body outlet end at its right end, the outer diameter of the inner tube connector engagement end is slightly smaller than the inner diameter of the inner tube connector, allowing it to be sleeved and secured therein, the inner tube body is longer than the outer tube body.

The stone removal basket comprises a basket tube body and a basket extending from the distal end of the basket tube body, the outer diameter of the basket tube body is slightly smaller than the inner diameter of the inner tube body, allowing it to move freely within the inner tube body, the basket tube body is longer than the inner tube body, the basket has a bag-shaped mesh structure forming several basket openings.

The stone-crushing rod comprises a handle ring at its left end and an elongated stone-crushing rod body extending distally, with a rod bending zone formed near its distal end, the outer diameter of the rod body is slightly smaller than the inner diameter of the inner tube body, allowing it to move freely within the inner tube body, the rod body is longer than the inner tube body.

In accordance to the above description, the present invention has the following beneficial effects and advantages:

By using the present invention, interventional radiologist can first perform bile duct/gallbladder drainage on the patient. After a one-day rest, the drainage channel will be fully expanded. The present invention can be inserted into the bile duct/gallbladder via the expanded drainage channel after another day of resting. At this point, the stone removal basket of the present invention can be positioned at the location of the stone through the inner tube and the outer tube. The stone can be captured by the basket, and by fixing the catheter and pulling the basket containing the stone outward, the stone can be crushed into smaller size and easily to be removed. If a stone cannot be crushed by tightening the basket, the stone-crushing rod can be employed to break the stone into fragments by force. The stone fragments can then be removed from the body using the basket.

During the procedure, the surgent can easily create a stone removal channel and perform the stone removal process using only local anesthesia and sedatives using the present invention. Patients with cardiopulmonary diseases who cannot undergo general anesthesia can still have the stone removal procedures. The entire stone removal process can be performed in an outpatient clinic without the need for admission to the hospital. For patients with gallbladder stones, all stones within the gallbladder can be completely removed, allowing the gallbladder to be preserved and avoiding the issue of fat digestion disorders associated with gallbladder removal. For stones in the left intrahepatic bile duct, the use of the stone removal catheter enables stone removal without the need for liver resection surgery. The stone removal catheter procedure has no age restrictions and no limitations on the size of the stones.

Many of the attendant features and advantages of the present invention will become better understood with reference to the following detailed description considered in connection with the accompanying figures and drawings.

### BRIEF DESCRIPTION OF THE DRAWINGS

The steps and the technical means adopted by the present invention to achieve the above and other objects can be best understood by referring to the following detailed description of the preferred embodiments and the accompanying drawings.

FIG. 1 illustrates a preferred embodiment of the outer tube and the inner tube in accordance to the present invention.

FIG. 2A illustrates a preferred embodiment of the connector of the outer tube and the connector of the inner tube in accordance to the present invention.

FIG. 2B illustrates a preferred embodiment of the rounded arc closure at the terminal openings of the outlet end of the outer tube body and the inner tube body in accordance to the present invention.

FIG. 3 illustrates a preferred embodiment of the stone removal basket and the stone-crushing rod in accordance to the present invention.

FIG. 4 illustrates an assembly schematic of several preferred embodiments in accordance to the present invention.

FIG. 5 illustrates an operational schematic of several preferred embodiments in accordance to the present invention.

### DETAILED DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts. It is not intended to limit the method by the exemplary embodiments described herein. In the following detailed description, for purposes of explanation, numerous specific details are set forth in order to attain a thorough understanding of the disclosed embodiments. It will be apparent, however, that one or more embodiments may be practiced without these specific details. As used in the description herein and throughout the claims that follow, the meaning of “a”, “an”, and “the” may include reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to.

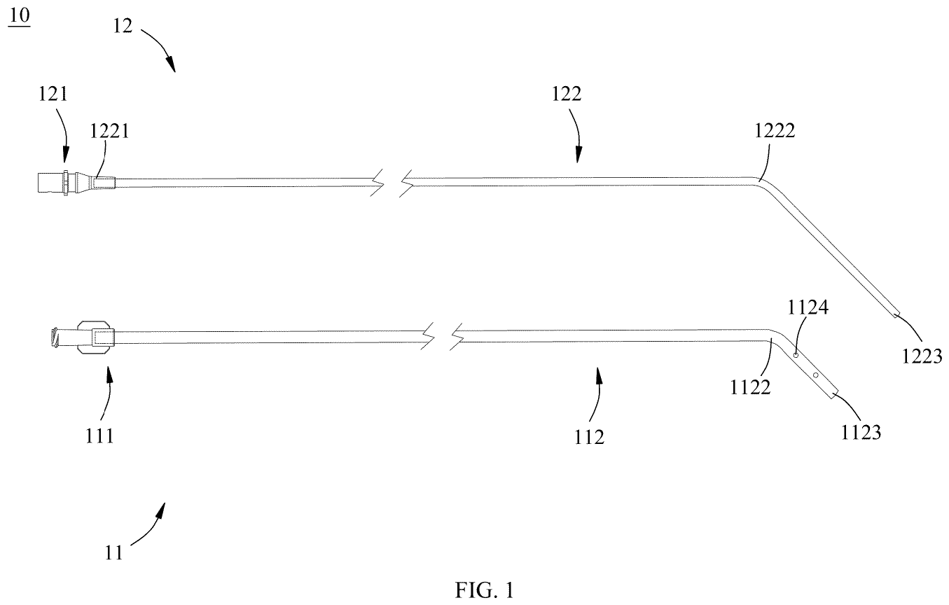

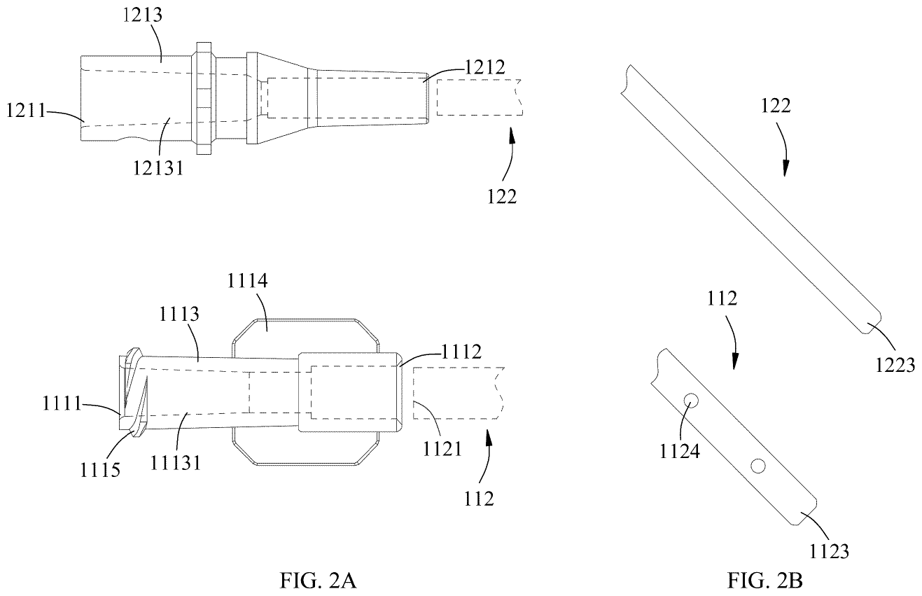

With reference to FIGS. 1, 2A, and 2B, a stone removal device 10 of the present invention comprises an outer tube 11, an inner tube 12 configured to be sleeved within the outer tube 11, and a stone removal basket 13 and/or a stone-crushing rod 14 configured to be sleeved within the inner tube 12.

### Outer Tube>

The outer tube 11 comprises an outer tube connector 111 and an outer tube body 112 that is detachably fixed to the outer tube connector 111. As shown in the lower part of FIG. 2A, the outer tube connector 111 includes a first outer tube opening 1111 on its left side and a second outer tube opening 1112 on its right side. The first outer tube opening 1111 and the second outer tube opening 1112 are elongated and connected as an outer tube column 1113.

The outer tube column 1113 features an outer tube inner passage 11131 that extends from the first outer tube opening 1111 to the second outer tube opening 1112, with two sections of differing inner diameters. The inner diameter of the outer tube inner passage 11131 on the first outer tube opening 1111 side is slightly smaller than that on the second outer tube opening 1112 side. An outer wall on the first outer tube opening 1111 side is equipped with an outer tube thread 1115. On the second outer tube opening 1112 side, an outer wall of the outer tube column 1113 is provided with a handheld tab 1114, which extends outwardly from both sides of the outer tube column 1113 in a sheet-like configuration.

The outer tube body 112 is a long, slender tube. As shown in FIGS. 1, 2A, and 2B, its left end includes an outer tube connector engagement end 1121, which extends longitudinally to the right. At the right end of the outer tube body 112, there is an outer tube bending zone 1122, which continues for a short distance before reaching an outer tube body outlet 1123. The outer diameter of the outer tube connector engagement end 1121 is slightly smaller than the inner diameter of the outer tube inner passage 11131 of the outer tube column 1113 at the second outer tube opening 1112, allowing it to be sleeved and secured within the second outer tube opening 1112. The design of the outer tube bending zone 1122 provides greater flexibility, allowing the present invention to penetrate deeper into the patient's body at various angles.

Preferably, the outer tube body 112 is provided with at least one fluid suction hole 1124 between the outer tube bending zone 1122 and the outer tube body outlet 1123. This enables excess bodily fluids to be suctioned during the stone removal process. The terminal opening at the outer tube body outlet 1123 is a rounded arc closure. The material of the outer tube body 112 is preferably slightly harder than the hardness of human tissue, allowing it to penetrate into the body more effectively and reach the target site. At the same time, the rounded arc closure at the terminal opening of the outer tube body outlet 1123 minimizes the risk of damaging surrounding tissue during the procedure.

### <Inner Tube>

The inner tube 12 comprises an inner tube connector 121 and an inner tube body 122 that is detachably fixed to the inner tube connector 121.

As shown in an upper part of FIG. 2A, the inner tube connector 121 includes a first inner tube opening 1211 on the left side and a second inner tube opening 1212 on the right side. The first inner tube opening 1211 extends in the direction of the second inner tube opening 1212, forming an inner tube column 1213. The inner tube column 1213 features an inner passage 12131 that extends from the first inner tube opening 1211 to the second inner tube opening 1212. The inner passage 12131 has two sections with different inner diameters, where the inner diameter on the first inner tube opening 1211 side is slightly smaller than the inner diameter on the second inner tube opening 1212 side. These two sections are connected by a tapered transition. An outer wall of the first inner tube opening 1211 side has an inner tube thread (not shown in the figures). The second inner tube opening 1212 of the inner tube column 1213 can be sleeved within the first outer tube opening 1111 of the outer tube connector 111 allowing the inner tube connector 121 to engage and combine with the outer tube connector 111.

Preferably, in another preferred embodiment of the present invention, the inner tube connector 121, near the second inner tube opening 1212, includes a sleeve (not shown in the figures) that is rotatably surrounded on an exterior of the inner tube connector 121. This sleeve is a regular component from a standard specification of the conventional stone removal devices and can be implemented with or without in the present invention.

The inner tube body 122 extends as a long, slender tube. As shown in FIG. 1, its left end includes an inner tube connector engagement end 1221, which extends longitudinally to the right. At the right end of the inner tube body 122, there is an inner tube bending zone 1222, which continues for a short distance before reaching an inner tube outlet 1223. The outer diameter of the inner tube connector engagement end 1221 is slightly smaller than the inner diameter of the inner tube connector 121, allowing it to be sleeved and fixed securely within the inner tube connector 121. The length of the inner tube body 122 is longer than the outer tube body 112, so when the inner tube body 122 is sleeved within the outer tube body 112, the inner tube outlet 1223 can extend beyond the outer tube body 112. The design of the inner tube bending zone 1222 provides greater flexibility, allowing the present invention to penetrate or reach into deeper body at various angles.

At a terminal of the inner tube outlet 1223 is a rounded arc closure. A hardness of a material of the inner tube body 122 is preferably slightly harder than a hardness of human tissue, enabling it to penetrate into the patient's body more effectively and reach the target site. Additionally, the rounded arc closure at the terminal of the inner tube outlet 1223 minimizes the risk of damaging surrounding human tissue during the surgery procedure.

Furthermore, the outer tube 11, including the outer tube connector 111 and the outer tube body 112, as well as the inner tube 12, including the inner tube connector 121 and the inner tube body 122, can be made of colorless or colored polyvinyl chloride (PVC), polycarbonate (PC), or polyethylene (PE). Preferably, to facilitate visibility during procedures within the body, especially for the outer tube body 112 and the inner tube body 122, the material may contain a contrast agent, such as barium sulfate, allowing the present invention to be visible under imaging equipment like X-ray machines. In some preferable embodiment, all components of the present invention possess antibacterial, antiviral, or even antifungal properties, ensuring cleanliness during use and a longer shelf life.

### <Stone Removal Basket>

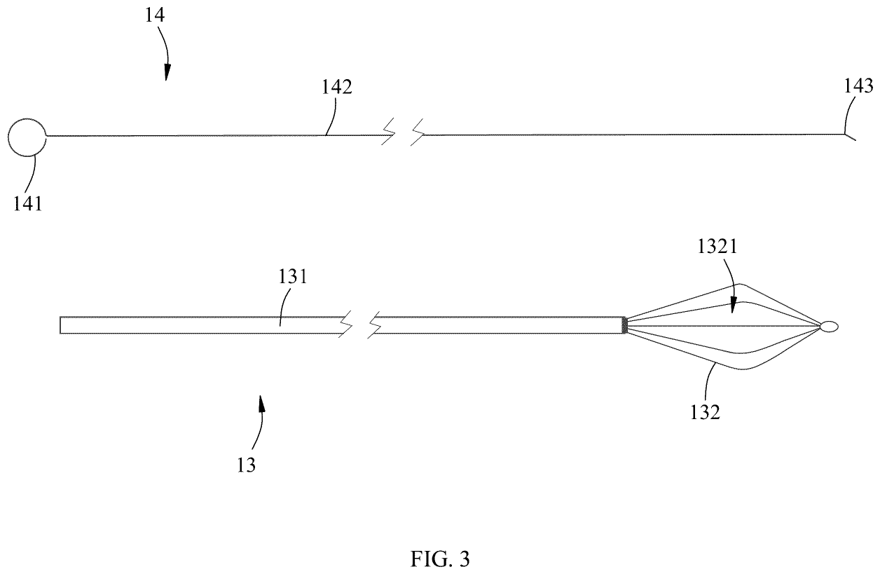

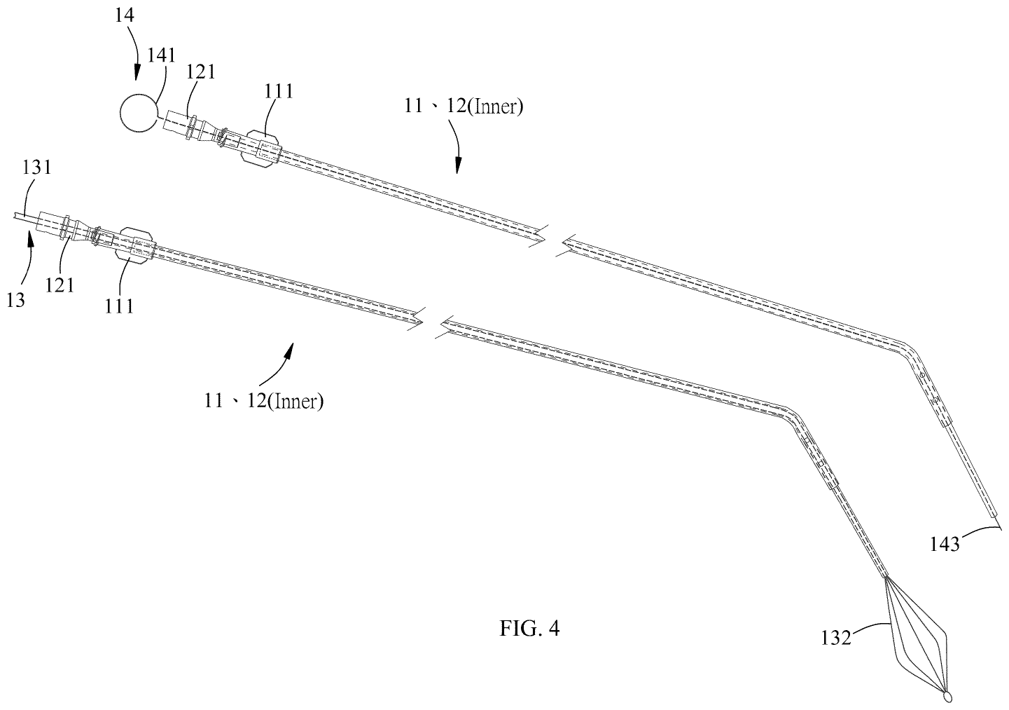

With reference to FIGS. 3 and 4, the stone removal basket 13, as shown in a lower part of FIG. 3, comprises a basket tube body 131 and a basket 132 extending from a distal end of the basket tube body 131. An outer diameter of the basket tube body 131 is slightly smaller than the inner diameter of the inner tube body 122, allowing it to move freely within the inner tube body 122. Additionally, the length of the basket tube body 131 is longer than that of the inner tube body 122. As a result, when the basket tube body 131 is inserted into the inner tube body 122, the basket 132 can protrude from the inner tube outlet 1223. The basket 132 is a bag-shaped mesh with several basket openings 1321. A material of the basket 132 is preferably a flexible or elastic metal, such as stainless steel wire.

### <Stone-Crushing Rod>

The stone-crushing rod 14 includes a handle ring 141 at its proximal end and an elongated stone-crushing rod 142 extending distally, with a rod bending zone 143 formed near its distal end. The outer diameter of the stone-crushing rod 142 is slightly smaller than the inner diameter of the inner tube body 122, allowing it to move freely within the inner tube body 122. Additionally, the length of the stone-crushing rod 142 is longer than that of the inner tube body 122. When the stone-crushing rod 142 is inserted into the inner tube body 122, the rod bending zone 143 can protrude from the inner tube outlet 1223. The stone-crushing rod 14 is preferably made of stainless steel.

With reference to FIG. 4, a preferred embodiment of the present invention shows the stone removal device 10 with the stone removal basket 13 or the stone-crushing rod 14 installed. During assembly, the inner tube body 122 is inserted through the inner tube outlet 1223 into the outer tube body 112 from the first outer tube opening 1111 of the outer tube connector 111. The inner tube outlet 1223 protrudes from the outer tube body 112, and the inner tube connector 121 is secured to the outer tube connector 111. Depending on the need during the procedure of the surgery, the stone removal basket 13 or the stone-crushing rod 14 can then be inserted into the inner tube body 122 through the inner tube connector 121. Since both the basket tube body 131 and the rod body 142 are longer than the inner tube body 122, the basket 132 and the rod bending zone 143 of the rod can protrude from the inner tube body 122.

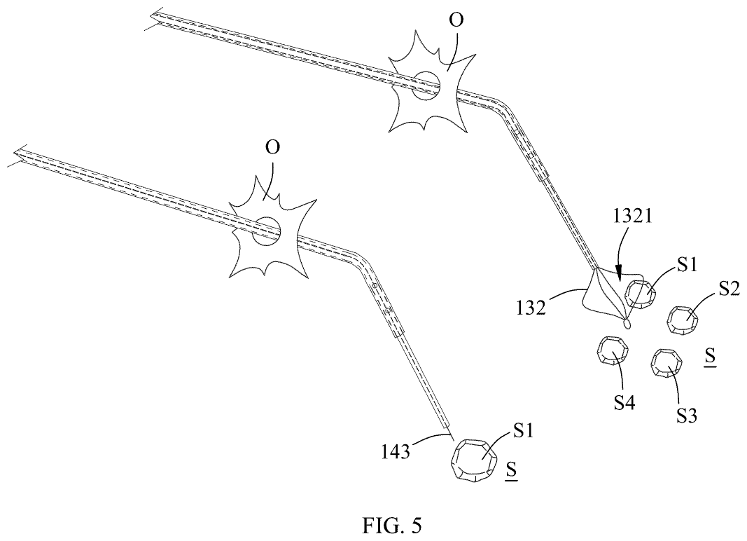

FIG. 5 illustrates a preferred embodiment of an operational flow of the stone removal device 10. It is used for retrieving and removing stones within the body, including but not limited to stones in the gallbladder or bile duct.

During the surgery, the outer tube 11 is first introduced through a surgical opening O to a location near the target stone site S within the patient's body, such as the gallbladder or bile duct, which may contain multiple stones S1 (S2, S3, S4 . . . ). The material of the outer tube body 112 is preferably slightly harder than human tissue, enabling it to penetrate the body and reach the target site effectively and easily. Additionally, the rounded arc closure at the terminal opening of the outer tube body outlet 1123 minimizes the risk of damaging human tissue during the procedure.

Next, the inner tube body 122 is inserted through the inner tube outlet 1223 into the outer tube body 112 from the first outer tube opening 1111 of the outer tube connector 111. The inner tube outlet 1223 protrudes beyond the outer tube body 112 and is positioned closer to the stone site S. The inner tube connector 121 is then secured to the outer tube connector 111.

If the stones S1 are small enough to fit directly into the basket 132, the stone removal basket 13 could be used to remove the stone S1 directly. The stone removal basket 13 is inserted into the inner tube body 122 through the inner tube connector 121, allowing the basket 132 to protrude from the inner tube outlet 1223 and contact the stone S1. The stone S1 is captured within the basket 132 through the basket openings 1321. By pulling the basket tube body 131 outward, the basket 132 tightens around the stone S1, pressing it against the inner tube outlet 1223. Continued pulling of the basket tube body 131 causes the stone S1 to fracture into smaller pieces due to the tightening force of the basket 132. This process is repeated until the fragments of stone S1 are smaller than the inner diameter of the inner tube body 122, after which the basket 132 is used to extract the fragments from the body, completing the stone removal process. Since the materials of the inner tube body 122 and the outer tube body 112 are of sufficient hardness without harming human tissue, they maintain their structure and provide support during the procedure, preventing deformation when pulling the basket tube body 131.

On the other hand, if the size of the stone S1 is too big or too hard, the stone-crushing rod 14 can be firstly used to break the stone S1 into smaller piece. The handle ring 141 is held to manipulate the rod bending zone 143 of the stone-crushing rod 142, which is used to break the stone S1 into smaller pieces. Once the stone S1 has been broken into smaller fragments, the basket 132 then can be used to remove the fragments from the body, completing the stone removal process.

The above specification, examples, and data provide a complete description of the present disclosure and use of exemplary embodiments. Although various embodiments of the present disclosure have been described above with a certain degree of particularity, or with reference to one or more individual embodiments, those with ordinary skill in the art could make numerous alterations or modifications to the disclosed embodiments without departing from the spirit or scope of this disclosure.

What is claimed is:

1. A stone removal device, comprising:
an outer tube, an inner tube configured to be sleeved within the outer tube, and a stone removal basket and/or a stone-crushing rod configured to be sleeved within the inner tube; wherein the outer tube comprises an outer tube connector and an outer tube body detachably fixed to the outer tube connector;
the outer tube connector includes a first outer tube opening at its left end and a second outer tube opening at its right end, the first outer tube opening and the second outer tube opening are connected by an outer tube column, and an outer tube thread is provided on the outer wall of the first outer tube opening;
the outer tube body is a long slender tube with an outer tube connector engagement end at its left end, extending longitudinally to the outer tube body outlet at its right end, the outer diameter of the outer tube connector engagement end is slightly smaller than the inner diameter of the outer tube inner passage within the second outer tube opening of the outer tube column, allowing it to be sleeved and secured therein;
the inner tube comprises an inner tube connector and an inner tube body detachably fixed to the inner tube connector;
the inner tube connector includes a first inner tube opening at its left end and a second inner tube opening at its right end, the first inner tube opening extends towards the second inner tube opening, forming an inner tube column, an inner tube thread is provided on the outer wall of the first inner tube opening, the inner tube column of the second inner tube opening is configured to be sleeved within the first outer tube opening of the outer tube connector;
the inner tube body is a long slender tube with an inner tube connector engagement end at its left end, extending longitudinally to the inner tube body outlet end at its right end, the outer diameter of the inner tube connector engagement end is slightly smaller than the inner diameter of the inner tube connector, allowing it to be sleeved and secured therein, the inner tube body is longer than the outer tube body;
the stone removal basket comprises a basket tube body and a basket extending from the distal end of the basket tube body, the outer diameter of the basket tube body is slightly smaller than the inner diameter of the inner tube body, allowing it to move freely within the inner tube body, the basket tube body is longer than the inner tube body, the basket has a bag-shaped mesh structure forming several basket openings; and
the stone-crushing rod comprises a handle ring at its left end and an elongated stone-crushing rod body extending distally, with a rod bending zone formed near its distal end, the outer diameter of the rod body is slightly smaller than the inner diameter of the inner tube body, allowing it to move freely within the inner tube body, the rod body is longer than the inner tube body.

2. The stone removal device as claimed in claim 1, wherein:
the outer tube column comprises an outer tube inner passage extending from the first outer tube opening to the second outer tube opening, with two sections of differing inner diameters, where the inner diameter on the first outer tube opening side is slightly smaller than that on the second outer tube opening side; and
the inner tube column comprises an inner passage extending from the first inner tube opening to the second inner tube opening, with two sections of differing inner diameters, where the inner diameter on the first inner tube opening side is slightly smaller than that on the second inner tube opening side, connected by a tapered transition.

3. The stone removal device as claimed in claim 1, wherein: the outer side of the outer tube column near the second outer tube opening has a handheld tab extending outwardly in a sheet-like form.

4. The stone removal device as claimed in claim 1, wherein:
the right end of the outer tube body includes an outer tube bending zone, extending a distance to the outer tube body outlet;
the right end of the inner tube body includes an inner tube bending zone, extending a distance to the inner tube body outlet end;
when the inner tube body is sleeved within the outer tube body, the inner tube body outlet end protrudes beyond the outer tube body; and
the right end of the stone-crushing rod includes a rod bending zone that can protrude beyond the inner tube body.

5. The stone removal device as claimed in claim 1, wherein: the terminal opening of the outer tube body outlet and/or the inner tube body outlet end is a rounded arc closure.

6. The stone removal device as claimed in claim 4, wherein: a fluid suction hole is configured to the outer tube body between the outer tube bending zone and the outer tube body outlet.

7. The stone removal device as claimed in claim 1, wherein:
the outer tube and the inner tube is colorless or colored polyvinyl chloride (PVC), polycarbonate (PC), or polyethylene (PE); and
the stone removal basket and the stone-crushing rod are stainless steel.

8. The stone removal device as claimed in claim 7, wherein: a material of the outer tube body and/or the inner tube body contains a contrast agent.

9. The stone removal device as claimed in claim 1, wherein: when the stone removal basket is sleeved within the inner tube body and the outer tube body, pulling the basket tube body does not cause the inner tube body and the outer tube body to bend.
